(19) United States
(12) Patent Application Publication
(10) Pub. No.: US 2022/0406454 A1
(43) Pub. Date: Dec. 22, 2022

(54) 3D-PRINTABLE TELEMEDICINE DEVICE

(71) Applicant: INTERNATIONAL BUSINESS MACHINES CORPORATION, Armonk, NY (US)

(72) Inventors: Bo Wen, New York, NY (US); Bing Dang, Chappaqua, NY (US); Vince Siu, Thornhill (CA); Noel Christopher Codella, White Plains, NY (US); Erhan Bilal, Westport, NY (US); Jeffrey L. Rogers, Briarcliff Manor, NY (US)

(21) Appl. No.: 17/304,183

(22) Filed: Jun. 16, 2021

(51) Int. Cl. G16H 40/67; G16H 40/40; G16H 80/00; G06F 30/10; A61B 7/04; G09B 19/00

(52) U.S. Cl. CPC G16H 40/67; G16H 40/40; G16H 80/00; G06F 30/10; A61B 7/04; G09B 19/003; G06F 2119/18

(57) ABSTRACT

A method, a structure, and a computer system for enabling telemedicine using printed devices. Exemplary embodiments may include receiving a design for a device and printing the device based on the design using a printer. The exemplary embodiments may further include combining the device with a smart device and utilizing the device to collect data during a telemedicine session administered on the smart device.

### BACKGROUND

The exemplary embodiments relate generally to telemedicine, and more particularly to 3D-printable telemedicine devices.

Unnecessary Emergency Room (ER) visits lead to increased ER demand, resulting in an overall reduced quality of care and additional transfer of pathogens. Telemedicine, i.e., virtual doctor visits, can tackle this problem by allowing physicians to remotely evaluate and diagnose patients. Currently, however, the use of telemedicine may be limited by several factors, the biggest of which may be access to necessary medical equipment.

### SUMMARY

The exemplary embodiments disclose a method, a structure, and a computer system for enabling telemedicine using printed devices. Exemplary embodiments may include receiving a design for a device and printing the device based on the design using a printer. The exemplary embodiments may further include combining the device with a smart device and utilizing the device to collect data during a telemedicine session administered on the smart device.

### BRIEF DESCRIPTION OF THE SEVERAL VIEWS OF THE DRAWINGS

The following detailed description, given by way of example and not intended to limit the exemplary embodiments solely thereto, will best be appreciated in conjunction with the accompanying drawings, in which:

FIG. 1 depicts an exemplary schematic diagram of a 3D-printable telemedicine device system 100, in accordance with the exemplary embodiments.

FIG. 2 depicts an example configuration of the 3D-printable device telemedicine system 100, in accordance with the exemplary embodiments.

FIG. 3 depicts an exemplary flowchart 300 illustrating the operations of a 3D-printable telemedicine device program 142 of the 3D-printable device telemedicine system 100, in accordance with the exemplary embodiments.

FIG. 4A-C depicts an example illustration of the 3D-printable telemedicine device 110, in accordance with the exemplary embodiments.

FIG. 5 depicts an exemplary block diagram depicting the hardware components of the 3D-printable telemedicine device system 100 of FIG. 1, in accordance with the exemplary embodiments.

FIG. 6 depicts a cloud computing environment, in accordance with the exemplary embodiments.

FIG. 7 depicts abstraction model layers, in accordance with the exemplary embodiments.

The drawings are not necessarily to scale. The drawings are merely schematic representations, not intended to portray specific parameters of the exemplary embodiments. The drawings are intended to depict only typical exemplary embodiments. In the drawings, like numbering represents like elements.

### DETAILED DESCRIPTION OF THE SEVERAL VIEWS OF THE DRAWINGS

Detailed embodiments of the claimed structures and methods are disclosed herein; however, it can be understood that the disclosed embodiments are merely illustrative of the claimed structures and methods that may be embodied in various forms. The exemplary embodiments are only illustrative and may, however, be embodied in many different forms and should not be construed as limited to the exemplary embodiments set forth herein. Rather, these exemplary embodiments are provided so that this disclosure will be thorough and complete, and will fully convey the scope to be covered by the exemplary embodiments to those skilled in the art. In the description, details of well-known features and techniques may be omitted to avoid unnecessarily obscuring the presented embodiments.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the interest of not obscuring the presentation of the exemplary embodiments, in the following detailed description, some processing steps or operations that are known in the art may have been combined together for presentation and for illustration purposes and in some instances may have not been described in detail. In other instances, some processing steps or operations that are known in the art may not be described at all. It should be understood that the following description is focused on the distinctive features or elements according to the various exemplary embodiments.

Unnecessary Emergency Room (ER) visits lead to increased ER demand, resulting in an overall reduced quality of care and additional transfer of pathogens. Telemedicine, i.e., virtual doctor visits, can tackle this problem by allowing physicians to remotely evaluate and diagnose patients. Currently, however, the use of telemedicine may be limited by several factors, the biggest of which may be access to necessary medical equipment.

In fact, many devices and most machines are not practical nor available to a patient at home. Consider a stethoscope used to screen patients for acute respiratory syndromes, a procedure known as auscultation. Traditional mechanical stethoscopes cannot be used in telemedicine application, while digital stethoscopes are designed for high end medical usage, making them very expensive.

The present invention provides a readily available, low cost solution to the current problems in telemedicine via use of mechanical, 3D-printed device. These devices, when used in conjunction with a smart device and one or more auxiliary components as needed, allow a physician to remotely evaluate and diagnose a patient using devices and machines previously limited to an ER or physician's office. While a 3D-printed device may be designed for any applicable procedure, an example use case of the present invention is described using a 3D-printed device for coupling a stethoscope to a cell phone microphone. Using an application on the cell phone, the patient can connect with a physician who can instructs a user how to use the 3D-printed device as well as evaluate acoustic signals of the patient's heart and lungs. In addition, the acoustic signals can be recorded and stored for later analysis, e.g., by a physician or artificial intelligence algorithms.

The 3D-print design of the device can be open sourced and distributed via internet. All the other required auxiliary components (e.g., headphone, microphone, smart device, etc.) are all commonly found household items. This ensures accessibility and scalability, allowing anyone to create the device at home or at a convenient location, e.g., work. Moreover, embodiments of the present invention provide the advantage of administering telemedicine in remote and less accessible locations via the ability to develop, fabricate, and utilize necessary medical devices using as little as access to a 3D printer and internet connection. The 3D-printed devices can be tested prior to use in order to ensure the 3D-printed device, and overall setup, meets the quality standard required for medical diagnosis. The test may be performed, e.g., while configuring software associated with the 3D-printed device.

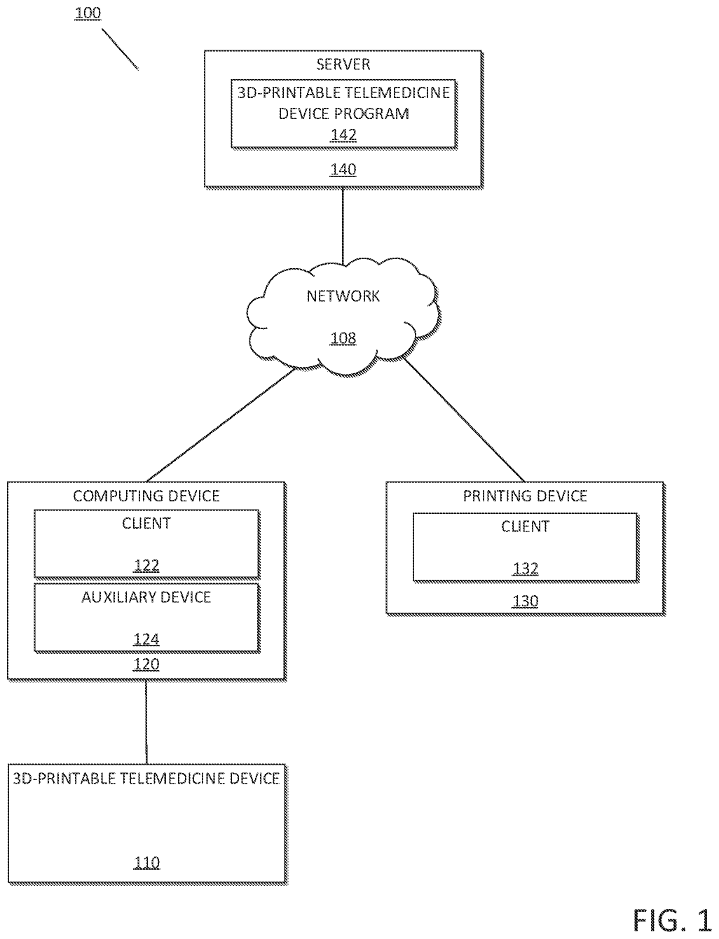

FIG. 1 depicts the 3D-printable device telemedicine system 100, in accordance with exemplary embodiments. According to the exemplary embodiments, the 3D-printable device telemedicine system 100 may include a 3D-printable telemedicine device 110, a computing device 120, a printing device 130, and a server 140, which all may be interconnected via a network 108. While programming and data of the exemplary embodiments may be stored and accessed remotely across several servers via the network 108, programming and data of the exemplary embodiments may alternatively or additionally be stored locally on as few as one physical computing device or amongst other computing devices than those depicted.

In the exemplary embodiments, the network 108 may be a communication channel capable of transferring data between connected devices. In the exemplary embodiments, the network 108 may be the Internet, representing a worldwide collection of networks and gateways to support communications between devices connected to the Internet. Moreover, the network 108 may utilize various types of connections such as wired, wireless, fiber optic, etc., which may be implemented as an intranet network, a local area network (LAN), a wide area network (WAN), or a combination thereof. In further embodiments, the network 108 may be a Bluetooth network, a Wi-Fi network, or a combination thereof. The network 108 may operate in frequencies including 2.4 GHz and 5 GHz internet, near-field communication, Z-Wave, Zigbee, etc. In yet further embodiments, the network 108 may be a telecommunications network used to facilitate telephone calls between two or more parties comprising a landline network, a wireless network, a closed network, a satellite network, or a combination thereof. In general, the network 108 may represent any combination of connections and protocols that will support communications between connected devices.

In exemplary embodiments, the 3D-printable telemedicine device 110 may be a device fabricated via printing machine, e.g., a three-dimensional (3D) printing machine. In embodiments, the 3D-printable telemedicine device 110 may be a resin, plastic, metal, thermoplastic, polycarbonate, carbon fiber, graphene, combination thereof, etc. The designs for the 3D-printable telemedicine device 110 may be open sourced and/or developed by private entities such as medical facilities and labs. As such, the designs may be available for general download or distributed on an as needed basis by prescribing entities. Such designs may be peer reviewed, rated, validated in professional lab environment, etc., before being distributed by a service provider (e.g., via the network 108). The designs may further include clear instructions on printing material choice and parameter configuration, as will be described in greater detail, and conform to common 3D printing file formats such as stereolithography (.stl). In embodiments, the 3D-printable telemedicine device 110 may be designed for fabrication in small scale 3D-printing machines, e.g., those available for residential or remote use. To that point, the 3D-printable telemedicine device 110 may be designed at small scale or, e.g., printed in connectable segments such that versions of the 3D-printable telemedicine device 110 larger than the printing device 130 may still be fabricated.

In exemplary embodiments, the computing device 120 may include the client 122 and an auxiliary device 124, and may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. While the computing device 120 is shown as a single device, in other embodiments, the computing device 120 may be comprised of a cluster or plurality of computing devices, in a modular manner, etc., working together or working independently.

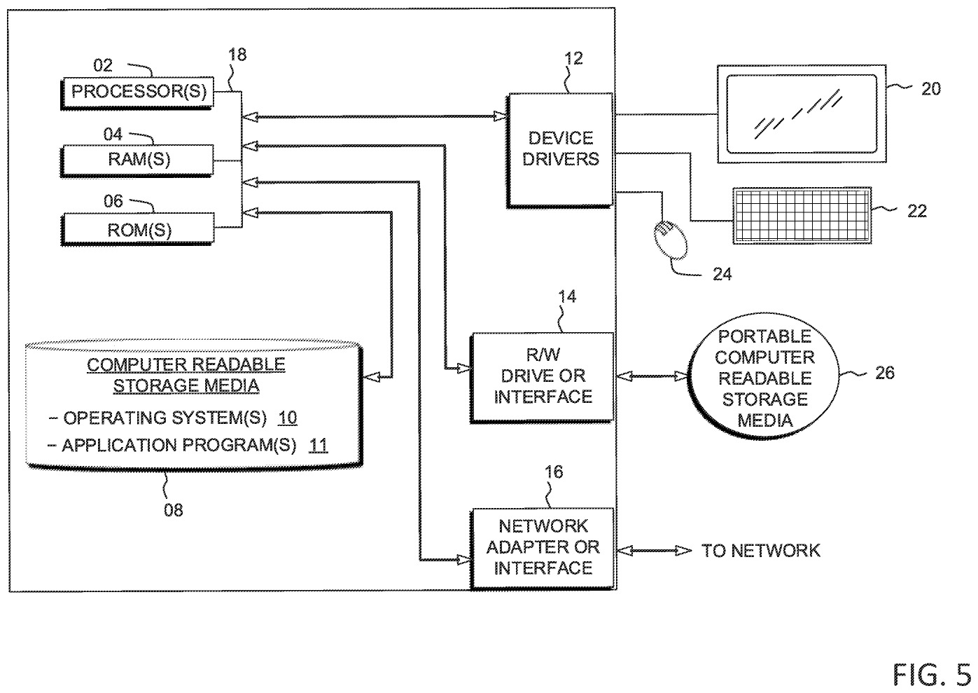

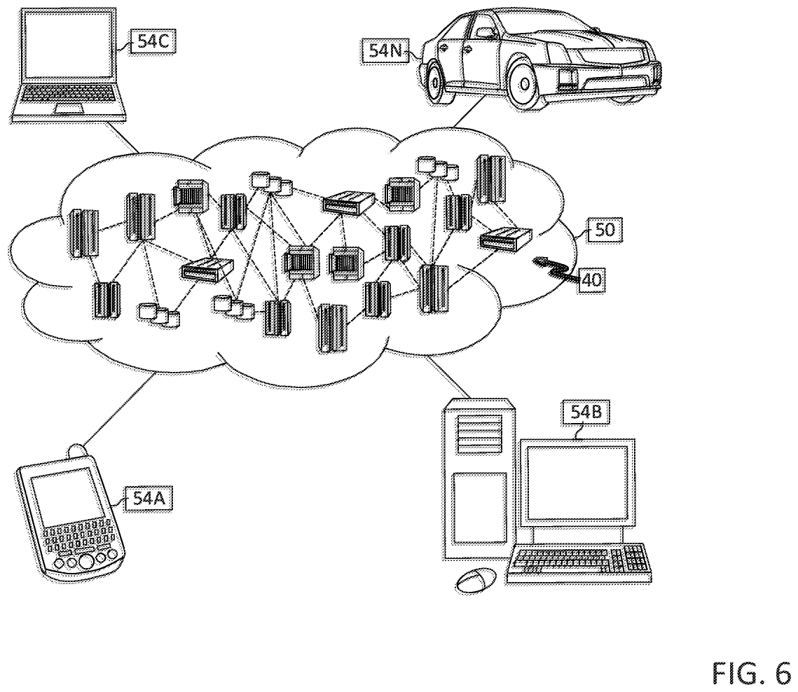

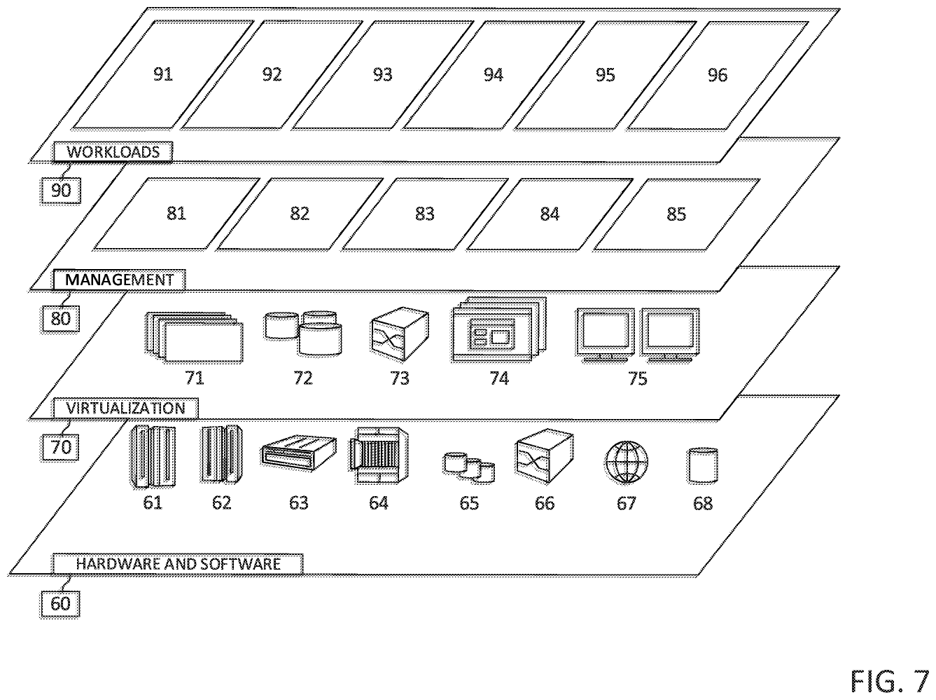

In exemplary embodiments, the computing device 120 may include one or more devices capable of collecting data, for example to capture video data, audio data, activity/movement data, biometric data, etc. Accordingly, the computing devices 120 may comprise mono/color cameras, infrared cameras, depth-sensing cameras/sensors, light detection and ranging (Lidar), microphones, accelerometers, gyroscopes, pressure sensors, etc., capable of collecting color images, thermal images, object/environment depth, sound, velocity, acceleration, etc. Moreover, the data collected by the computing device 120 may be transmitted to another device, e.g., the server 140, via the network 108. The computing device 120 is described in greater detail as a hardware implementation with reference to FIG. 5, as part of a cloud implementation with reference to FIG. 6, and/or as utilizing functional abstraction layers for processing with reference to FIG. 7.

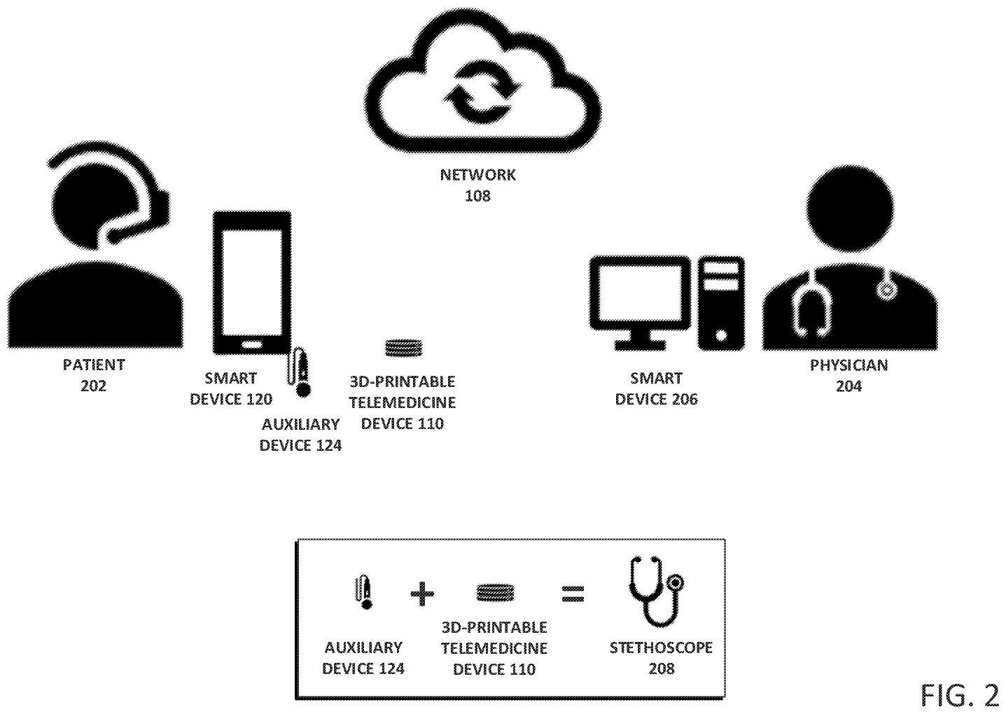

In exemplary embodiments, the client 122 may act as a client in a client-server relationship with a server, for example the server 140, and may be a software and/or hardware application capable of communicating with and providing a user interface for a user to interact with a server and other computing devices via the network 108. Moreover, in the example embodiment, the client 122 may be capable of transferring data between the computing device 120 and other devices via the network 108. In embodiments, the client 122 may utilize various wired and wireless connection protocols for data transmission and exchange, including Bluetooth, 2.4 GHz and 5 GHz internet, near-field communication, etc. The client 122 is described in greater detail with respect to FIG. 2-7.

In exemplary embodiments, the auxiliary device 124 may improve the computing device 120 and include one or more components such as speakers, headphones, microphones, earbuds, sensors, etc. For example, the auxiliary device 124 may, e.g., enhance a use of or increase a capability of the computing device 120. As such, the auxiliary device 124 may be a headphone, microphone, etc. In addition, the auxiliary device 124 may connect and transmit data to the computing device 120 and other devices via wired or wireless connections over the network 108, e.g., Wi-Fi, Bluetooth, etc.

In exemplary embodiments, the printing device 130 may include the client 132, and may be any device capable of printing an ink, plastic, resin, etc. onto a sacrificial or non-sacrificial substrate. The printing device 130 may be designed for commercial or residential use, and implement printing techniques such as Fused Deposition Modeling (FDM), Stereolithography (SLA), Digital Light Processing (DLP), Selective Laser Sintering (SLS), Selective Laser Melting (SLM), Electron Beam Melting (EMB), Laminated Object Manufacturing (LOM), Binder Jetting (BJ), and Material Jetting/Wax Casting, etc. Accordingly, and based on purpose, the printing device 130 may vary in features such as size, ink type, required post-processing, resource consumption, etc., which all may be considered when choosing a design of the 3D-printable telemedicine device 110. In general, however, the printing device 130 may be any suitable device capable of printing 3D objects. In order to transmit and receive communications, the printing device 130 may further include an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. While the printing device 130 is shown as a single device, in other embodiments, the printing device 130 may be comprised of a cluster or plurality of computing devices, in a modular manner, etc., working together or working independently. The printing device 130 is described in greater detail as a hardware implementation with reference to FIG. 5, as part of a cloud implementation with reference to FIG. 6, and/or as utilizing functional abstraction layers for processing with reference to FIG. 7.

In exemplary embodiments, the client 132 may act as a client in a client-server relationship with a server, for example the server 140, and may be a software and/or hardware application capable of communicating with and providing a user interface for a user to interact with a server and other computing devices via the network 108. Moreover, in the example embodiment, the client 132 may be capable of transferring data between the printing device 130 and other devices via the network 108. In embodiments, the client 132 may utilize various wired and wireless connection protocols for data transmission and exchange, including Bluetooth, 2.4 GHz and 5 GHz internet, near-field communication, etc. The client 132 is described in greater detail with respect to FIG. 2-7.

In exemplary embodiments, the server 140 includes a 3D-printable telemedicine device program 142, and may act as a server in a client-server relationship with a client, e.g., the client 122 and 132. The server 140 may be an enterprise server, a laptop computer, a notebook, a tablet computer, a netbook computer, a personal computer (PC), a desktop computer, a server, a personal digital assistant (PDA), a rotary phone, a touchtone phone, a smart phone, a mobile phone, a virtual device, a thin client, an IoT device, or any other electronic device or computing system capable of sending and receiving data to and from other computing devices. While the server 140 is shown as a single device, in other embodiments, the server 140 may be comprised of a cluster or plurality of computing devices, in a modular manner, etc., working together or working independently. The server 140 is described in greater detail as a hardware implementation with reference to FIG. 5, as part of a cloud implementation with reference to FIG. 6, and/or as utilizing functional abstraction layers for processing with reference to FIG. 7.

In embodiments, the 3D-printable telemedicine device program 142 may be a software and/or hardware program that may receive a configuration and print a device. The 3D-printable telemedicine device program 142 may further calibrate the printed device, as well as aid in performing a procedure using the device. The 3D-printable telemedicine device program 142 may additionally determine whether the procedure was performed correctly and, if so, store and analyse the results of the procedure. The 3D-printable telemedicine device program 142 is described in greater detail with reference to FIG. 2-7.

FIG. 2 depicts an example configuration of the 3D-printable device telemedicine system 100, in accordance with the exemplary embodiments. As illustrated by FIG. 2, the 3D-printable device telemedicine system 100 is being used to perform an auscultation, and comprises a patient 202 operating the smart device 120 in connection with a physician 204 on a smart device 206 via the network 108. The smart device 120 is connected to a microphone as the auxiliary device 124, which may be further connected to the 3D-printable telemedicine device 110. In this example embodiment, the 3D-printable telemedicine device 110 fits around the auxiliary device 124 (microphone) and is designed to guide audio waves towards the auxiliary device 124 in the same manner a stethoscope guides audio waves towards an ear of the physician 204. In this way, the combination of the auxiliary device 124 and the 3D-printable telemedicine device 110 work together in order to act as a stethoscope 208, both remotely from the physician 204 and without the need for the stethoscope 208. Moreover, once configured and as applicable, the patient 202 can continue to utilize the 3D-printable telemedicine device 110 without intervention by the physician 204.

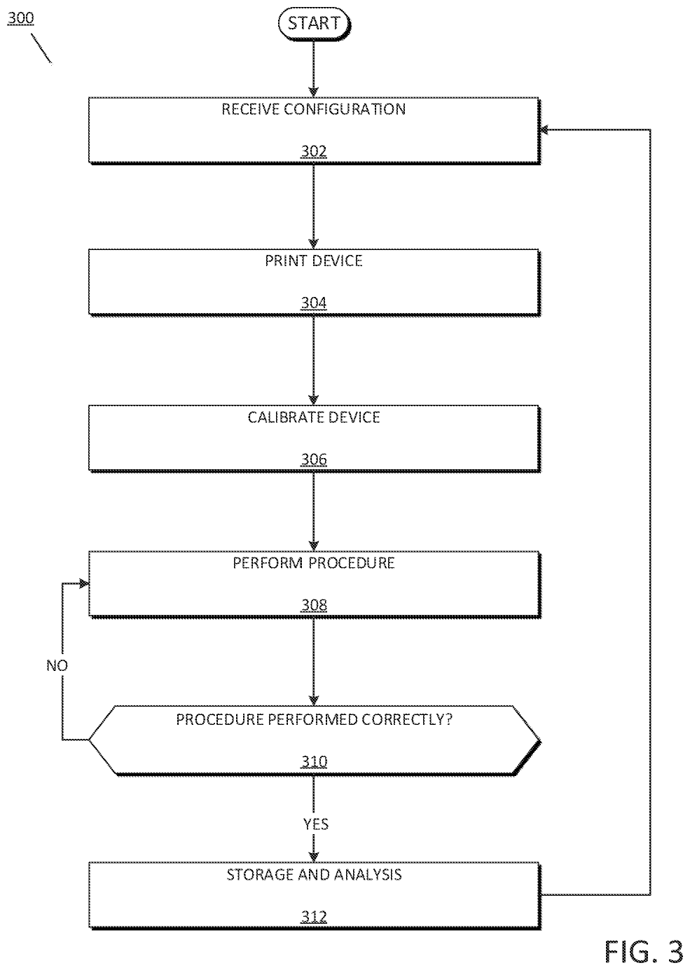

FIG. 3 depicts an exemplary flowchart 300 illustrating the operations of a 3D-printable telemedicine device program 142 of the 3D-printable device telemedicine system 100, in accordance with the exemplary embodiments. The operations of the 3D-printable telemedicine device program 142 will be described in conjunction with FIG. 3, as well as FIG. 2.

The 3D-printable telemedicine device program 142 may receive a configuration (step 302). In embodiments, the 3D-printable telemedicine device program 142 may receive a configuration detailing the patient 202, one or more procedures needed thereby, and the 3D-printable telemedicine device 110 required to perform such procedures. The 3D-printable telemedicine device program 142 may be configured for a particular patient 202, e.g., through use of profiles, and may receive patient information via reference to an electronic health/medical record, user/administrative input, one or more sensors, etc.

In embodiments, the 3D-printable telemedicine device program 142 may identify the procedures needed by the patient 202 based on user input, e.g., received from the patient 202/physician 204/administrator/etc. In more advanced embodiments, the 3D-printable telemedicine device program 142 may be configured to perform procedures at set intervals, e.g., once a day/week, or may be configured to automatically identify and implement one or more procedures using machine learning techniques. Such techniques may include identifying patterns in the patient or similarly situated patients and conforming a procedure regiment to such patterns. Moreover, using the machine learning techniques, best practices and most efficient procedures and regimens may be selected, and subjective patient conditions may be further incorporated into such machine learning analyses. In such embodiments, the 3D-printable telemedicine device program 142 may make use of machine learning techniques that identify and quantity patterns within large amounts of training data in one or more predictive models. The training data may be population data comprising a random selection of individuals, a specific cohort to which a patient belongs, a combination of both, etc., and the 3D-printable telemedicine device program 142 may generate the one or more models through machine learning techniques such as regression. In embodiments, the 3D-printable telemedicine device program 142 may be configured to implement both telemedicine procedures, i.e., with physician 204/administrator intervention, as well as those the patient 202 may perform themselves without physician 204/administrator intervention, for example measuring blood glucose or blood pressure. In embodiments implementing telemedicine, the patient 202 may be audially or visually assisted in performing the required procedure(s) by the physician 204 or administrator via the computing device 120 and the network 108, as will be described in greater detail forthcoming.

Receiving the configuration may further include receiving data for printing the 3D-printable telemedicine device 110 required to perform the procedures needed by the patient 202. The data may comprise a file enumerating the printing specifications of the 3D-printable telemedicine device 110 in a standard file format, e.g., stereolithography (.stl), and detail size, material, resolution, temperature, printing orientation, etc. In addition, the data may further include printer-specific fine tunings that, when used, result in a highest quality and greatest efficacy version of the 3D-printable telemedicine device 110. The fine tunings may depend on a brand and/or model of the printing device 130, and may be identified through trial and error, clinical testing, crowd sourcing of user feedback, etc. The fine tunings may be open source such that users such as patients, administrators, physicians, etc. can provide feedback, such as rankings, until the most effective fine tunings are identified for each brand and/or model of the printing device 130.

Similar to identifying necessary procedures above, the 3D-printable telemedicine device program 142 may utilize machine learning to provide a user a recommended configuration and fine tunings based on procedure needed, the printing device 130 available, etc. In embodiments, the 3D-printable telemedicine device program 142 may be configured to customize the design of the 3D-printable telemedicine device 110 based on patient considerations, such as patient size, weight, restrictions, disfigurements, etc. The 3D-printable telemedicine device program 142 may, e.g., print a smaller version of the 3D-printable telemedicine device 110 for patients identified as children, etc.

In order to better illustrate the operations of the 3D-printable telemedicine device program 142, reference is made to the example illustrated by FIG. 2 wherein the 3D-printable telemedicine device program 142 is used by the patient 202 to perform an auscultation. In this example, the patient 202 calls their physician 204 to complain of slight difficulty breathing and although the physician 204 does not have availability for the patient to come into the office the same day, the physician 204 is able to schedule a brief telemedicine visit for later that day. The physician then securely transmits a file to the patient 202 detailing the printed device 110 that allows the patient to use their smart phone and a microphone to act as the stethoscope 208 needed to perform the auscultation.

The 3D-printable telemedicine device program 142 may print the 3D-printable telemedicine device 110 (step 304). In embodiments, the 3D-printable telemedicine device program 142 may print the 3D-printable telemedicine device 110 based on the received device design using the printing device 130 that is accessible to and/or selected by the patient 202. The printing device 130, e.g., may be within a residence of the patient 202, a work location, a remote location, etc., and the 3D-printable telemedicine device program 142 may be configured to maintain a repository detailing the public and/or otherwise accessible printing devices 150 for a patient to select for printing the 3D-printable telemedicine device 110. The 3D-printable telemedicine device program 142 may, e.g., suggest one or more printing devices 130 to the patient 202 based on proximity to a current or future location, and preferred/default printing devices 150 may be stored in a patient profile.

In order to print the 3D-printable telemedicine device 110, the 3D-printable telemedicine device program 142 may ascertain, via communication with the printing device 130 or user input, the brand/model, compatibilities, capabilities, resources, etc. of the selected printing device 150. The 3D-printable telemedicine device program 142 may, e.g., detect a maximum size in cartesian coordinates, maximum resolution, temperature settings, available resources/ink, print speed, available duration to print, etc. The 3D-printable telemedicine device program 142 may further schedule the printing of the 3D-printable telemedicine device 110, e.g., immediately, as soon as possible, at a scheduled time, etc., such that the patient 202 may simply pick up the 3D-printable telemedicine device 110.

Returning to the illustrative example introduced above, the 3D-printable telemedicine device program 142 communicates with the printing device 130 within a home of the patient 202 via the 3D-printable telemedicine client 132 in order to receive the file detailing the 3D-printable telemedicine device 110. As the printing device 130 was previously configured, the received file includes fine tunings such as settings, parameters, and printing instructions specific to the brand and model of the printing device 130. Based on the received file, the printing device 130 prints the corresponding stethoscope adapting device.

The 3D-printable telemedicine device program 142 may calibrate the 3D-printable telemedicine device 110 (step 306). Calibrating the 3D-printable telemedicine device 110 may depend on the type of and procedure required by the 3D-printable telemedicine device 110, however calibration may generally include assembling the 3D-printable telemedicine device 110 and ensuring that the device 110 operates with sufficient quality, reliability, efficacy, etc. To that point, the 3D-printable telemedicine device program 142 may be configured to play instructional video, audio, or text to first assemble the 3D-printable telemedicine device 110, as well as run calibration and testing protocols upon finishing the assembly. Such calibration and testing protocols may involve simulating the desired procedure into the assembled 3D-printable telemedicine device 110 and ensuring the simulated procedure is replicated with sufficient accuracy, signal, etc. by the 3D-printable telemedicine device program 142. Simulating the desired procedure may be achieved, e.g., by playing pre-recorded sounds into the 3D-printable telemedicine device 110 when acting to receive audio, displaying image or video into the 3D-printable telemedicine device 110 when acting to receive video, etc. Based on the calibration, the patient 202 may then need modify/reprint the 3D-printable telemedicine device 110 or modify a manner in which it is used during the telemedicine session. It should be noted that some embodiments of the 3D-printable telemedicine device 110 may not require assembly, calibration, or, in some cases, auxiliary devices 124. For example, some embodiments of the 3D-printable telemedicine device 110 may adapt directly to the smart device 120, while in other embodiments, additional auxiliary devices 124 may be necessary.

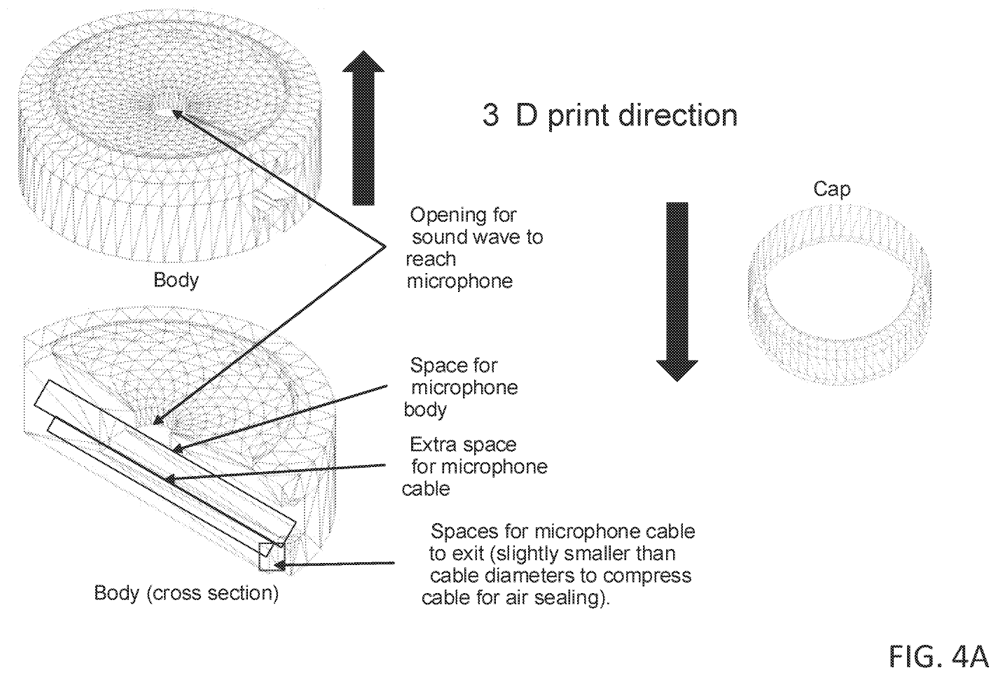

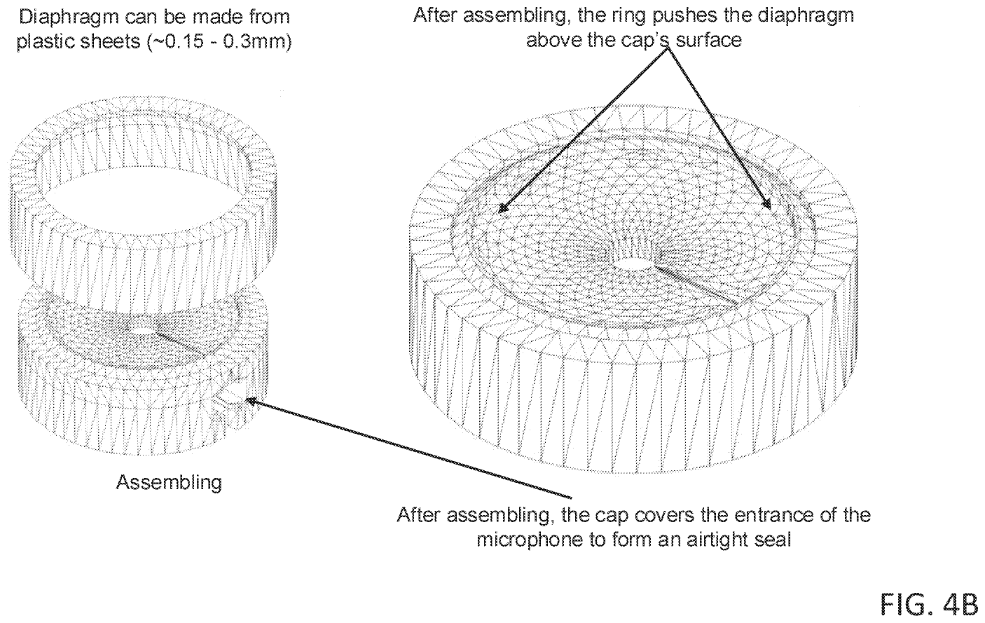

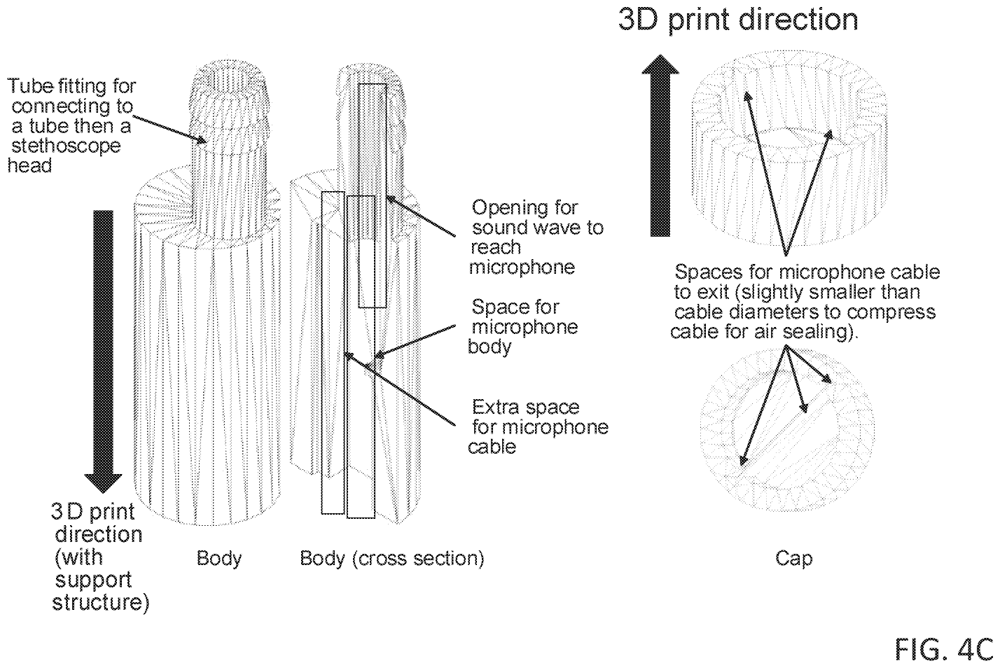

Furthering the previously introduced example, and with additional reference to FIG. 4, the patient 202 installs the 3D-printable telemedicine device 110 by encompassing an inline microphone of the auxiliary device 124 with the 3D-printable telemedicine device 110. The patient 202 may then record a segment of audio played by another computing device using the 3D-printable telemedicine device 110 and compare the output signal to the input signal in order to ensure the 3D-printable telemedicine device 110 is performing to sufficient standards.

The 3D-printable telemedicine device program 142 may perform the procedure using the 3D-printable telemedicine device 110 (step 308). In embodiments, the 3D-printable telemedicine device program 142 may enable the patient 202 to communicate with the physician 204 via the smart device 120, a computing device used by the physician 204, and the network 108. The physician 204 may assist the patient 202 in performing the procedure audially or visually, and such assistance may include both instructions for the patient to administer the procedure (e.g., where to place the device), as well as instructions for the patient 202 whilst receiving the procedure (e.g., to breathe deeply, exhale, etc.). With respect to the administration instructions, the physician 204 may direct the patient 202 to perform the procedure in a similar manner to that which the physician 204 would perform themselves, e.g., with respect to procedure location, configuration, duration, magnitude, etc. With respect to instructions for the patient 202 while receiving the procedure, the 3D-printable telemedicine device program 142 may similarly enable the physician 204 to instruct the patient 202 how to behave during the procedure, e.g., breathing exercises, muscle movements, describing a response to stimulation, etc., such that the telemedicine session mimics that of an in-person visit. Lastly, the 3D-printable telemedicine device program 142 allows for the transfer of data collected by the 3D-printable telemedicine device 110 and any auxiliary device 124 to the physician 204. Such data may be audio, visual, sensory, etc., and based on the procedure at hand.

With reference again to the previously introduced example and FIG. 3, the patient 202 performs an auscultation on themself with the assistance of the physician 204, and the 3D-printable telemedicine device program 142 records audio data gathered by the 3D-printable telemedicine device 110, the microphone auxiliary device 124, and the smart phone computing device 120.

In the exemplary embodiments, the 3D-printable telemedicine device program 142 may determine whether the procedure was performed correctly (decision 310). If, e.g., telemedicine was employed where the physician 204 or an administrator were present during the procedure, the 3D-printable telemedicine device program 142 may determine whether the procedure was correctly performed based on their user input. In such embodiments, the physician 204 or administrator may manually indicate or otherwise authorise the results of the procedure at a point during or following. Alternatively, in embodiments where telemedicine was not employed, the 3D-printable telemedicine device program 142 may determine whether the procedure was correctly performed by comparing the overall or periodic results of the procedure to an expected the results of the procedure in order to determine whether the results are within a tolerable range. Results of the comparison may then be transmitted to the physician 204, who may analyse the results to affirm, reject, change, or otherwise acknowledge the results of the procedure. Note that in some embodiments, e.g., merely precautionary procedures, the step of determining whether the procedure was correctly performed may be omitted.

If the 3D-printable telemedicine device program 142 determines that the procedure was not performed correctly (decision 210, “NO” branch), then the 3D-printable telemedicine device program 142 may be configured to perform the procedure once again. When it is determined that a procedure must be reperformed, the 3D-printable telemedicine device program 142 may make changes to the procedure such that performing the procedure successfully is more likely. For example, in embodiments where telemedicine was not employed, the 3D-printable telemedicine device program 142 may be configured to employ telemedicine in this instance to ensure the procedure is correctly performed. Alternatively, a more patient-friendly procedure may be selected when several are available, e.g., one that requires less patient dexterity or flexibility. In other embodiments, the patient 202 may be asked to see the physician 204 in an in-person visit. This may be due, for example, to the results of the procedure, a number of failed attempts at performing the procedure, etc. In such embodiments, the 3D-printable telemedicine device program 142 may be configured to reference the schedules of both the patient 202 and physician 204 in order to schedule a visit.

In the example above, for example, if the patient 202 fails to perform the auscultation successfully, the 3D-printable telemedicine device program 142 may ask that the patient 202 try again but hold the 3D-printable telemedicine device 110 in a different orientation.

If the 3D-printable telemedicine device program 142 determines that the procedure was performed correctly (decision 210, “NO” branch), then the 3D-printable telemedicine device program 142 may store and analyse the collected data (step 212).

In concluding the previously introduced example, the 3D-printable telemedicine device program 142 stores an audio recording of both the auscultation as well as any communication between the patient 202 and the physician 204.

One skilled in the art may appreciate that the 3D-printable telemedicine device program 142 may be advantageous in additional embodiments other than those illustrated. In an additional exemplary embodiment, e.g., the 3D-printable telemedicine device program 142 may be implemented in a remote location in which medical or other health supplies may be unavailable or delivery thereof unfeasible. The present invention may be employed in order to provide the necessary medical and/or health supplies based on the resources at hand within the remote site. In another exemplary embodiment, the mass need for a particular medical and/or health supply may cripple supplies thereof. The present invention may be employed in order to quickly mass produce the particular medical and/or health supply using any trusted resource with a printing machine, e.g., hospitals, universities, small businesses, etc. It will be appreciated that there are any number of use cases in which the present invention may prove advantageous, however the present disclosure only mentions several in the interest of brevity.

FIG. 4A-B depicts an example illustration of the 3D-printable telemedicine device 110, in accordance with the exemplary embodiments. In particular, FIG. 4A-B depict an embodiment in which the 3D-printable telemedicine device 110 comprises a stethoscope chest piece having a cap and a diaphragm. Here, the cap not only creates an airtight seal where the microphone is inserted, but also pushes the diaphragm outward to create a surface for pressing against the patient 202 and receiving/transferring audio signals. In this embodiment, the patient 202 need only the 3D-printable telemedicine device 110, the computing device 110 such as a smart phone, and the accessory 124 such as smart phone-connected ear bud in order to replicate a clinical-grade stethoscope at home.

FIG. 4C similarly depicts an example illustration of the 3D-printable telemedicine device 110 acting as a stethoscope, however here the patient 202 is already in possession of a stethoscope chest piece for connecting to the 3D-printable telemedicine device 110. Otherwise, the 3D-printable telemedicine device 110 similarly encompasses the microphone in a similar manner to that above.

FIG. 5 depicts a block diagram of devices used within the 3D-printable telemedicine device system 100 of FIG. 1, in accordance with the exemplary embodiments. It should be appreciated that FIG. 5 provides only an illustration of one implementation and does not imply any limitations with regard to the environments in which different embodiments may be implemented. Many modifications to the depicted environment may be made.

Devices used herein may include one or more processors 02, one or more computer-readable RAMs 04, one or more computer-readable ROMs 06, one or more computer readable storage media 08, device drivers 12, read/write drive or interface 14, network adapter or interface 16, all interconnected over a communications fabric 18. Communications fabric 18 may be implemented with any architecture designed for passing data and/or control information between processors (such as microprocessors, communications and network processors, etc.), system memory, peripheral devices, and any other hardware components within a system.

One or more operating systems 10, and one or more application programs 11 are stored on one or more of the computer readable storage media 08 for execution by one or more of the processors 02 via one or more of the respective RAMs 04 (which typically include cache memory). In the illustrated embodiment, each of the computer readable storage media 08 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a R/W drive or interface 14 to read from and write to one or more portable computer readable storage media 26. Application programs 11 on said devices may be stored on one or more of the portable computer readable storage media 26, read via the respective R/W drive or interface 14 and loaded into the respective computer readable storage media 08.

Devices used herein may also include a network adapter or interface 16, such as a TCP/IP adapter card or wireless communication adapter (such as a 4G wireless communication adapter using OFDMA technology). Application programs 11 on said computing devices may be downloaded to the computing device from an external computer or external storage device via a network (for example, the Internet, a local area network or other wide area network or wireless network) and network adapter or interface 16. From the network adapter or interface 16, the programs may be loaded onto computer readable storage media 08. The network may comprise copper wires, optical fibers, wireless transmission, routers, firewalls, switches, gateway computers and/or edge servers.

Devices used herein may also include a display screen 20, a keyboard or keypad 22, and a computer mouse or touchpad 24. Device drivers 12 interface to display screen 20 for imaging, to keyboard or keypad 22, to computer mouse or touchpad 24, and/or to display screen 20 for pressure sensing of alphanumeric character entry and user selections. The device drivers 12, R/W drive or interface 14 and network adapter or interface 16 may comprise hardware and software (stored on computer readable storage media 08 and/or ROM 06).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

Based on the foregoing, a computer system, method, and computer program product have been disclosed. However, numerous modifications and substitutions can be made without deviating from the scope of the exemplary embodiments. Therefore, the exemplary embodiments have been disclosed by way of example and not limitation.

It is to be understood that although this disclosure includes a detailed description on cloud computing, implementation of the teachings recited herein are not limited to a cloud computing environment. Rather, the exemplary embodiments are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 6, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 40 and cloud computing environment 50 can communicate with any type of computerized device over any type of network and/or network addressable connection (e.g., using a web browser).

Referring now to FIG. 7, a set of functional abstraction layers provided by cloud computing environment 50 (FIG. 6) is shown. It should be understood in advance that the components, layers, and functions shown in FIG. 7 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfilment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and 3D-printable telemedicine device program processing 96.

The exemplary embodiments may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A method for enabling telemedicine using printed devices, the method comprising:
receiving a design for a device;
printing the device based on the design using a printer;
combining the device with a smart device; and
utilizing the device to collect data during a telemedicine session administered on the smart device.

2. The method of claim 1, wherein the received design is based on at least one of a procedure required by a patient during the telemedicine session and specifications of the printer.

3. The method of claim 1, further comprising:
receiving instructions describing how to assembly and use the device during the telemedicine session.

4. The method of claim 1, further comprising:
calibrating the device by:
simulating the data collection with the device by receiving an output signal;
comparing the collected data with the output signal; and
adjusting the device based on the comparison.

5. The method of claim 1, wherein the device comprises a stethoscope chest piece and a housing for encompassing a microphone that is connected to the smart device.

6. The method of claim 5, wherein audio signals from a patient are passed through the device to the smart device via the microphone, and wherein to the audio signals are transmitted to a physician administering a remote auscultation via the telemedicine session.

7. The method of claim 1, further comprising:
recording the collected data for analysis.

8. A computer program product for enabling telemedicine using printed devices, the computer program product comprising:
one or more computer-readable storage media and program instructions stored on the one or more computer-readable storage media, the program instructions including a method, the method comprising:
receiving a design for a device;
printing the device based on the design using a printer;
combining the device with a smart device; and
utilizing the device to collect data during a telemedicine session administered on the smart device.

9. The computer program product of claim 8, wherein the received design is based on at least one of a procedure required by a patient during the telemedicine session and specifications of the printer.

10. The computer program product of claim 8, further comprising:
receiving instructions describing how to assembly and use the device during the telemedicine session.

11. The computer program product of claim 8, further comprising:
calibrating the device by:
simulating the data collection with the device by receiving an output signal;
comparing the collected data with the output signal; and
adjusting the device based on the comparison.

12. The computer program product of claim 8, wherein the device comprises a stethoscope chest piece and a housing for encompassing a microphone that is connected to the smart device.

13. The computer program product of claim 12, wherein audio signals from a patient are passed through the device to the smart device via the microphone, and wherein to the audio signals are transmitted to a physician administering a remote auscultation via the telemedicine session.

14. The computer program product of claim 8, further comprising:
recording the collected data for analysis.

15. A computer system for enabling telemedicine using printed devices, the computer system comprising:
one or more computer processors, one or more computer-readable storage media, and program instructions stored on one or more of the computer-readable storage media for execution by at least one of the one or more processors, the program instructions including a method, the method comprising:
receiving a design for a device;
printing the device based on the design using a printer;
combining the device with a smart device; and
utilizing the device to collect data during a telemedicine session administered on the smart device.

16. The computer system of claim 15, wherein the received design is based on at least one of a procedure required by a patient during the telemedicine session and specifications of the printer.

17. The computer system of claim 15, further comprising:
receiving instructions describing how to assembly and use the device during the telemedicine session.

18. The computer system of claim 15, further comprising:
calibrating the device by:
simulating the data collection with the device by receiving an output signal;
comparing the collected data with the output signal; and
adjusting the device based on the comparison.

19. The computer system of claim 15, wherein the device comprises a stethoscope chest piece and a housing for encompassing a microphone that is connected to the smart device.

20. The computer system of claim 19, wherein audio signals from a patient are passed through the device to the smart device via the microphone, and wherein to the audio signals are transmitted to a physician administering a remote auscultation via the telemedicine session.
